(19) United States
(12) Patent Application Publication
(10) Pub. No.: US 2026/0335510 A1
(43) Pub. Date: Oct. 29, 2026

(54) Cartilage Restoration System

(71) Applicant: Nolan Francis Chowansky, Bluffton, SC (US)

(72) Inventor: Nolan Francis Chowansky, Bluffton, SC (US)

(21) Appl. No.: 19/187,766

(22) Filed: Apr. 23, 2025

(51) Int. Cl. A61L 27/52; A61L 27/44; A61L 27/48

(52) U.S. Cl. CPC A61L 27/52; A61L 27/446; A61L 27/48; A61L 2400/06; A61L 2430/38

(57) ABSTRACT

The Cartilage Restoration System is an injectable hydrogel platform designed to permanently restore, reinforce, and replace fibrocartilaginous and cartilaginous tissues, including intervertebral discs, knee menisci, labra, and articular cartilage, for human and veterinary orthopedic applications, with potential non-orthopedic uses. The system comprises a biocompatible hydrogel precursor with synthetic or natural polymers, natural additives, crosslinking agents activated by thermal, chemical, photochemical, enzymatic, or ionic triggers, and reinforcing agents. Delivered minimally invasively via dual-chamber syringe, catheter, robotic-assisted system, or 3D-printed applicator, the hydrogel polymerizes in situ to form a non-degradable implant with a compressive strength of at least 3-5 MPa, three times that of native tissue. The implant resists degradation, ensures fatigue resistance, and integrates with native tissue through adhesive and bioactive components, sealing defects and supporting lifelong functionality. Modular configurations address disc regeneration, spinal fusion, cartilage repair, and osteochondral defects, with species-specific formulations for veterinary use.

### BACKGROUND OF THE INVENTION

### Field of the Invention

The present invention relates generally to biomaterials, biomedical implants, and tissue engineering within the field of regenerative medicine.

More specifically, it pertains to a permanent in situ injectable hydrogel system designed for the restoration, reinforcement, and replacement of fibrocartilaginous and cartilaginous tissues, including intervertebral discs, articular cartilage, knee menisci, labra (shoulder, hip, wrist), and related structures, with applications in orthopedic repair, spinal restoration, joint stabilization, spinal fusion, and veterinary orthopedic treatments, leveraging advanced biomaterial science to provide durable, load-bearing solutions exceeding natural tissue capacity.

### Description of Related Art

The field of orthopedic repair has advanced significantly with hydrogel-based systems, stem cell therapies, 3D-printed scaffolds, decellularized matrices, and metal-based implants targeting degenerative and traumatic damage to fibrocartilage and hyaline cartilage tissues, such as intervertebral discs, menisci, labra, and articular cartilage. Early patents, such as U.S. Pat. No. 5,047,055 (issued Sep. 10, 1991) and U.S. Pat. No. 5,976,186 (issued Nov. 2, 1999), describe hydrogel-based intervertebral disc nuclei designed to replace damaged disc tissue using degradable formulations that act as temporary scaffolds for tissue regeneration. Similarly, U.S. Patent Application Publication No. US20060093648A1 (published May 4, 2006) and U.S. Pat. No. 10,507,178 (issued Dec. 17, 2019) disclose in situ gelling hydrogels for disc degeneration and orthopedic repair, but these biodegradable systems produce repair tissue lacking the durability of native tissue and fail to provide immediate, long-term mechanical support, particularly in avascular tissues with limited regenerative capacity. Stem cell therapies, 3D-printed scaffolds, decellularized matrices, and metal-based implants, while promising, often require complex fabrication, face challenges in achieving immediate load-bearing capacity or permanent integration, or are limited by biocompatibility issues.

Unlike the Cartilage Restoration System, these prior arts do not offer a non-degradable implant with a compressive strength at least three times that of native tissue (e.g., 3-5 MPa vs. 1-1.5 MPa for native cartilage, validated by ASTM-compliant mechanical testing), nor do they employ multiple crosslinking methods (thermal, chemical, photochemical, enzymatic) or address veterinary applications.

Further developments in cartilage repair are evidenced by U.S. Pat. No. 9,393,195 (issued Jul. 19, 2016) and European Patent Application No. EP1773417A1 (published Apr. 18, 2007), which describe crosslinked polymer compositions for articular cartilage and meniscal repair, typically relying on biodegradable scaffolds. These systems are limited by donor site morbidity, poor integration under mechanical stress, and lack of permanent support, often resulting in suboptimal repair tissue. Japanese Patent No. JP2011517713A (published Jun. 16, 2011) introduces a non-degradable hydrogel for medical devices, but it does not specifically target fibrocartilage restoration or achieve a triple load-bearing capacity, nor does it cover applications like spinal fusion or veterinary orthopedic repair. In contrast, the Cartilage Restoration System provides a permanent, non-degradable implant with a compressive modulus of 3-5 MPa (vs. 1-1.5 MPa for native cartilage), validated by mechanical testing, and integrates adhesive and bioactive components for robust tissue bonding and defect sealing (e.g., annular tears), extending its utility to a broad range of anatomical sites and species.

A closely related advancement, U.S. Patent Application Publication No. US20120100103A1 (published Apr. 26, 2012), describes an in situ-forming injectable hydrogel using a dehydrogenation reaction between phenol or aniline moieties, enhanced by a water-soluble polymer linker (e.g., PEG), for applications including cartilage and intervertebral disc regeneration. While this system improves biostability and mechanical strength, it does not specify a triple load-bearing capacity, relies on a single crosslinking mechanism, and lacks the explicit non-degradable permanence of the present invention.

The Cartilage Restoration System distinguishes itself by combining multiple crosslinking methods (thermal, chemical, photochemical, enzymatic) to achieve rapid, versatile in situ polymerization, a validated triple load-bearing capacity, and modular applications (e.g., EternaSpine, Eterna Hyaline) for fibrocartilage repair, spinal fusion, joint stabilization, and veterinary uses. Unlike prior art, it offers immediate structural support, lifelong durability, and adaptability across diverse orthopedic challenges, addressing unmet clinical needs for enhanced biomechanical performance in both human and veterinary contexts.

### SUMMARY OF THE INVENTION

The Cartilage Restoration System is an injectable hydrogel system designed to permanently restore, reinforce, and replace fibrocartilaginous and cartilaginous tissues, including intervertebral discs, knee menisci, labra (shoulder, hip, wrist), and articular cartilage, for human and veterinary orthopedic applications.

The system comprises a flowable hydrogel precursor including a biocompatible base polymer (e.g., polyethylene glycol diacrylate at 10-15% w/v, poly(vinyl alcohol), polyurethane, or other synthetic or natural polymers capable of forming hydrogels), natural polymer additives (e.g., hyaluronic acid, collagen, chitosan at 1-2% w/v), crosslinking agents (0.1-0.5% w/v, activated by thermal, chemical, photochemical, enzymatic, ionic, or other methods), reinforcing agents (e.g., silica, microfibers, ceramic particles), and adhesive/bioactive components (e.g., catechol-functionalized polymers, TGF-β, or other growth factors). The hydrogel is delivered minimally invasively via a dual-chamber syringe, catheter, robotic-assisted system, 3D-printed applicator, or other delivery device, polymerizing in situ within minutes to form a non-degradable implant.

Polymerization occurs under physiologic conditions or with optional exogenous triggers (e.g., fiber optic light), achieving full strength in less than 30 minutes without requiring external energy sources beyond these triggers.

The cured hydrogel exhibits a compressive modulus of 3-5 MPa, as validated by mechanical testing (e.g., ASTM D695 at 1 mm/min strain rate) and finite element analysis, providing at least three times the compressive strength of native tissue (1-1.5 MPa for cartilage). Its non-degradable structure resists enzymatic, hydrolytic, and mechanical degradation for lifelong functionality, with a water content of 50-90% and tailored crosslink density to replicate the viscoelastic properties of native tissues while exceeding their biomechanical performance.

Adhesive and bioactive components facilitate chemical and mechanical bonding with native tissues, enabling sealing of defects, such as annular tears, and promoting cellular ingrowth for enhanced integration. The system supports applications including intervertebral disc regeneration, meniscal repair and reconstruction, labral repair (shoulder, hip, wrist triangular fibrocartilage complex), spinal fusion reinforcement, and osteochondral repair, with optional subchondral bone perforations for anchoring in hyaline cartilage applications.

The Cartilage Restoration System includes modular configurations: (1) a disc regeneration and spinal fusion module to restore disc height and seal annular tears; (2) a cartilage repair module for menisci, labra, and wrist triangular fibrocartilage complex, enhancing joint stability; and (3) a hyaline cartilage replacement module with hydrophilic side chains (e.g., polyethylene glycol, zwitterionic groups) for low-friction surfaces and triple load-bearing capacity.

Veterinary applications extend to repairing discs, cartilage, and menisci in animals, such as dogs and horses, using hydrogel formulations tailored to species-specific biomechanical and anatomical requirements. The system is delivered percutaneously or arthroscopically under image guidance (e.g., fluoroscopy, ultrasound), with a delivery apparatus featuring interchangeable tips and radiopaque agents for precise placement. By providing immediate structural support, long-term durability, and adaptability across diverse anatomical sites, the invention addresses limitations of prior art, such as biodegradable scaffolds and regenerative therapies, for improved orthopedic outcomes in human and veterinary medicine.

### BRIEF DESCRIPTION OF THE DRAWINGS

The following drawings illustrate exemplary embodiments and are not intended to limit the scope:

FIG. 1: A cross-sectional view of a damaged spinal disc and a normal spinal disc.

FIG. 2: A cross-sectional view of normal spinal disc with the cartilage restoration system hydrogel.

FIG. 3: A cross-sectional view of spinal disc that was damaged and repaired by the cartilage restoration system.

FIG. 4: A cross-sectional view of hyaline cartilage damaged, as well as healthy fibrocartilage.

FIG. 5: A cross-sectional view of prior-damaged hyaline cartilage receiving the cartilage restoration system post hydrogel injection.

FIG. 6: A cross-sectional view of fibrocartilage damaged.

FIG. 7: A cross-sectional view of fibrocartilage being restored with the fibrocartilage restoration system.

FIG. 8: A block diagram of the fibrocartilage restoration system for fibrocartilage and spinal discs.

FIG. 9: A block diagram of the fibrocartilage restoration system for hyaline cartilage system.

### DETAILED DESCRIPTION OF THE INVENTION

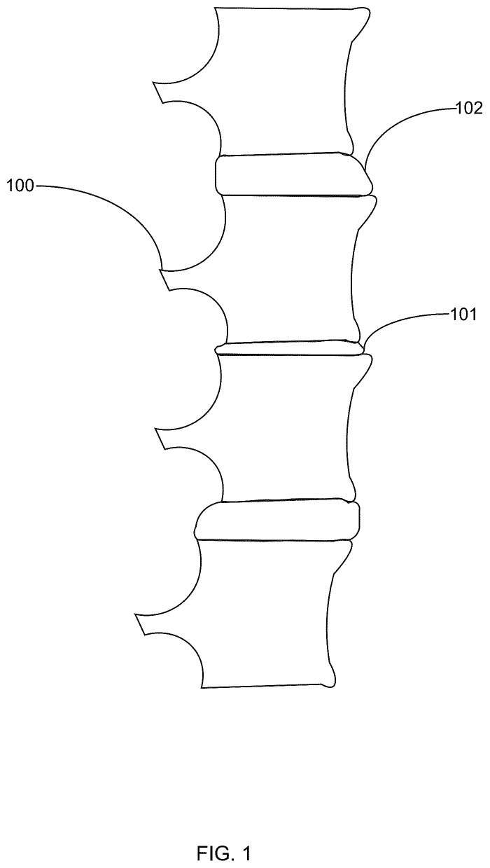

The Cartilage Restoration System is an injectable hydrogel platform designed to permanently restore, reinforce, and replace fibrocartilaginous and cartilaginous tissues, including intervertebral discs, knee menisci, labra of the shoulder, hip, and wrist, and articular cartilage, applicable to human and veterinary orthopedic repair and spinal fusion. The system comprises a biocompatible hydrogel precursor that polymerizes in situ to form a non-degradable implant with a compressive modulus of 3-5 MPa, approximately three times that of native tissue, ensuring superior load-bearing capacity. FIGS. 1 through 9 illustrate the system's components, mechanisms, and applications, with medical terminology defined to ensure clarity for medical professionals.

The hydrogel composition includes polyethylene glycol diacrylate (PEGDA) at 10-15% weight per volume, selected for biocompatibility, with alternative polymers like poly(vinyl alcohol) or polyurethane prepolymers, and natural additives such as hyaluronic acid or collagen at 1-2% to mimic the extracellular matrix. Crosslinking agents, activated by thermal, chemical (Michael addition), photochemical (photoinitiators at 0.1-0.5%), or enzymatic (transglutaminase) triggers, enable curing in seconds to minutes, while reinforcing agents like silica or collagen fibers enhance strength. The system is delivered via a dual-chamber syringe or catheter under fluoroscopy with radiopaque agents, curing in less than 30 minutes to bond with native tissue, maintaining 50-90% water content.

FIG. 1 illustrates a cross-sectional side view of the spine, comparing a damaged spinal disc (101) with reduced height and nucleus pulposus volume to a normal disc (102) with intact annulus fibrosus and hydration. Component (100) shows the spine's vertebral bodies, establishing the clinical need for repair in degenerative disc disease, where disc dehydration and structural failure cause pain. The terminology, including “nucleus pulposus” (gel-like core) and “annulus fibrosus” (fibrous ring), is precise, depicting the problem addressed.

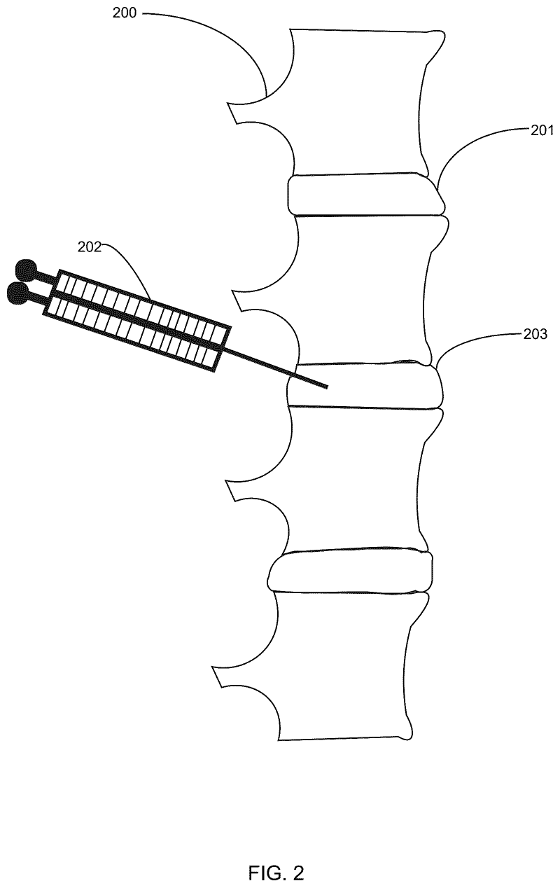

FIG. 2 depicts a normal spinal disc (201) receiving hydrogel (202) to prevent degeneration, shown in a side view of the spine (200). The hydrogel integrates with the nucleus pulposus, enhancing viscoelastic properties and compressive modulus for lifelong durability, as shown in the reinforced disc (203). This prophylactic application, with terms like “hydrogel” (hydrophilic polymer network) and “viscoelastic properties” (viscous and elastic behavior), clearly illustrates the preventive mode.

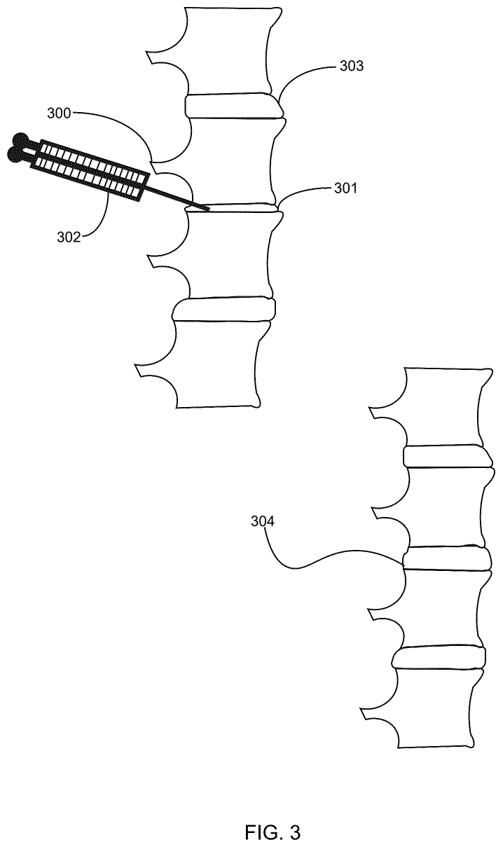

FIG. 3 shows a damaged disc (301) repaired via a double-barrel syringe (302) delivering hydrogel, restoring height and function (304) compared to a normal disc (303). The side view (300) illustrates minimally invasive delivery, with the hydrogel sealing annular tears and mimicking nucleus pulposus properties.

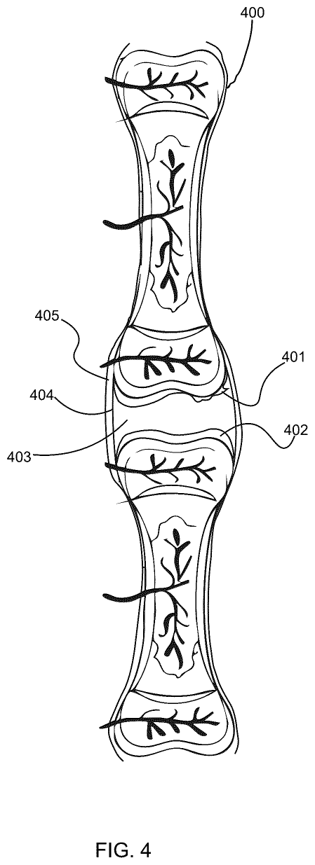

FIG. 4 depicts a joint (400) with damaged hyaline cartilage (401), showing erosion, contrasted with normal cartilage (402), alongside synovial fluid (403), ligament (404), and joint capsule (405). The damaged cartilage reflects osteoarthritis, while the normal cartilage provides a repair goal, with “hyaline cartilage” (smooth articular surface) and “synovial fluid” (joint lubricant) clearly defined. This figure illustrates the cartilage repair context.

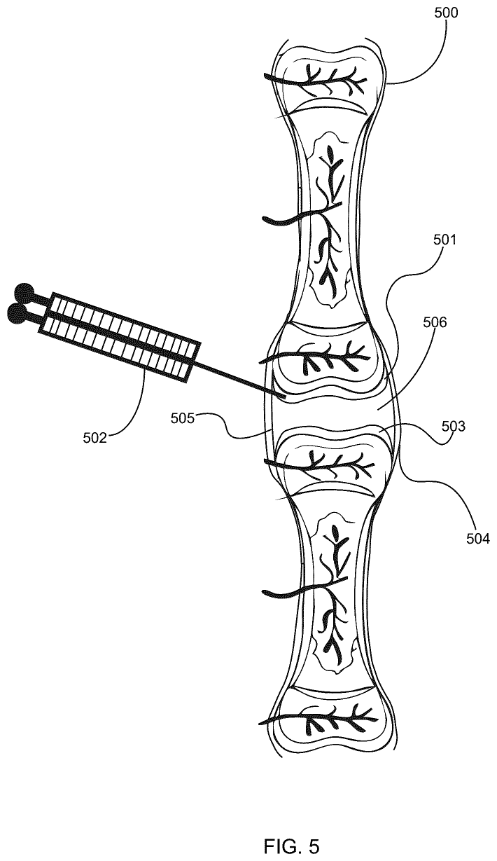

FIG. 5 shows damaged hyaline cartilage (501) receiving hydrogel (502) to form repaired hyaline cartilage, (503) shows healthy fibrocartilage and bone structure (500), with joint capsule (504), ligament (505), and synovial fluid (506). The hydrogel's low-friction surface mimics native cartilage, integrating with subchondral bone, with “arthroscopic injection” (minimally invasive delivery) ensuring clarity. The figure depicts the repair process and outcome.

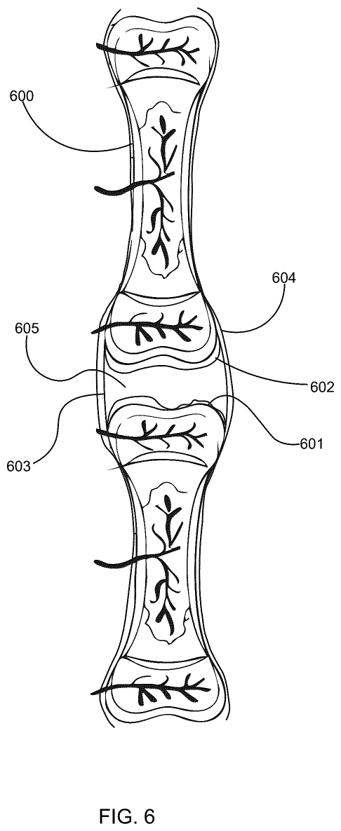

FIG. 6 illustrates a joint (600) with damaged fibrocartilage (601), such as a torn meniscus, compared to healthy hyaline cartilage (602), with ligament (603), joint capsule (604), and synovial fluid (605). The damaged fibrocartilage represents conditions like meniscal tears, with “fibrocartilage” (tough, fibrous tissue) accurately applied. This figure shows the pathological state addressed.

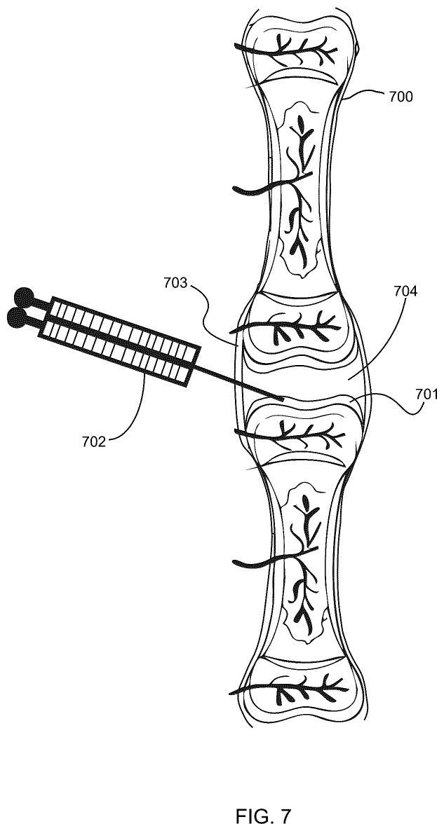

FIG. 7 depicts repaired fibrocartilage (701) from a damaged state (601) via hydrogel (702) in a joint (700), with joint capsule (703) and synovial fluid (704). The hydrogel's reinforcing fibers ensure hoop strength, stabilizing the joint, with “hoop strength” (resistance to radial expansion) clearly defined. The figure illustrates fibrocartilage restoration.

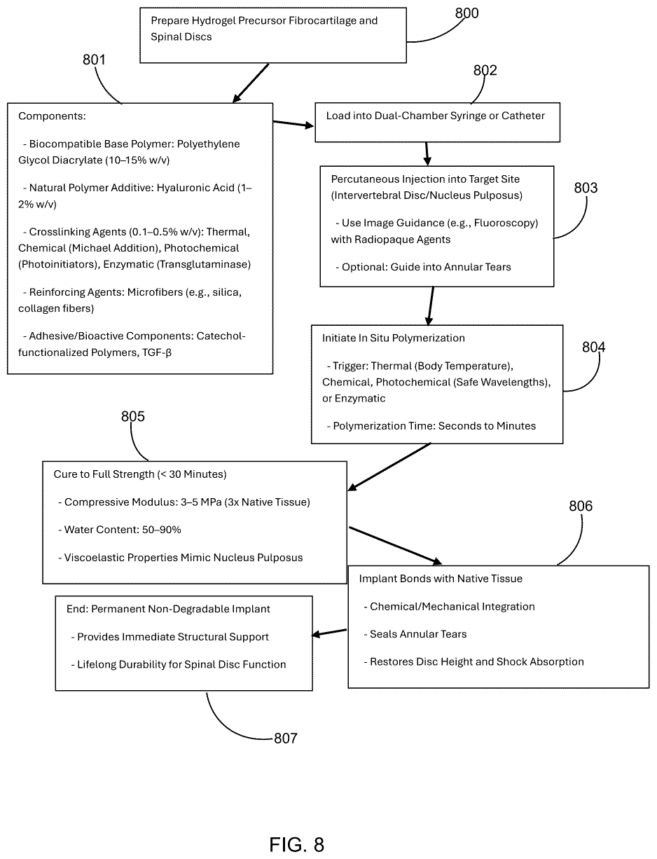

FIG. 8 outlines the system for fibrocartilage and spinal discs, starting with hydrogel preparation (800) using PEGDA, hyaluronic acid, and crosslinking agents (801), loaded into a dual-chamber syringe (802). Percutaneous injection (803) under fluoroscopy delivers the hydrogel, which polymerizes (804) and cures (805) to bond with tissue (806), forming a durable implant (807). Terms like “Michael addition” (chemical crosslinking) and “fluoroscopy” (real-time imaging) ensure clarity.

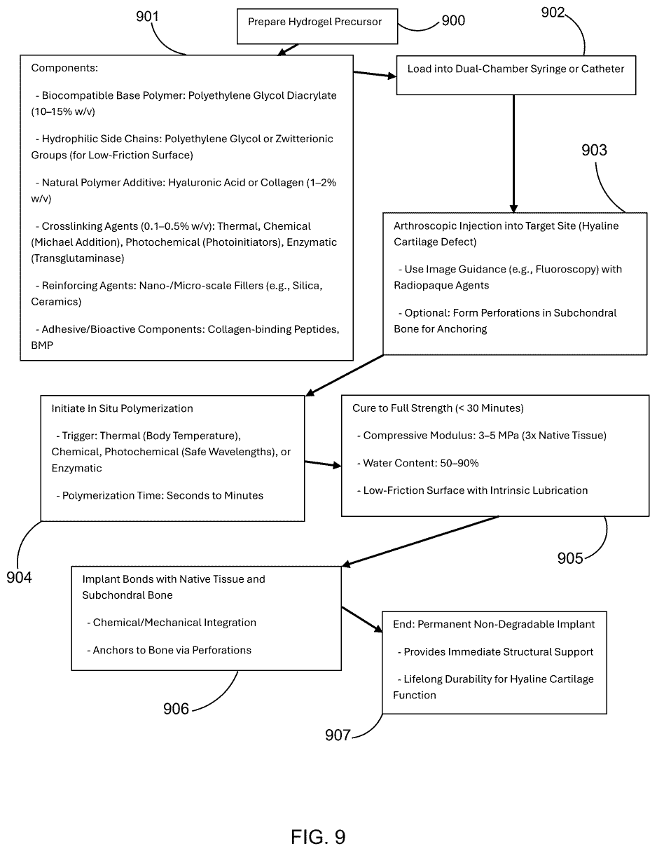

FIG. 9 details the hyaline cartilage system, preparing hydrogel (900) with PEGDA, zwitterionic groups, and collagen-binding peptides (901), delivered arthroscopically (902-903). The hydrogel polymerizes (904), cures to a low-friction surface (905), and bonds to subchondral bone (906), forming a permanent implant (907). Terminology like “zwitterionic groups” (low-friction molecules) and “subchondral bone” (bone beneath cartilage) is precise.

The disc regeneration module, shown in FIGS. 1-3 and 8, restores degenerated discs or reinforces healthy ones using PEGDA and photoinitiators, injected percutaneously. The cartilage repair module, depicted in FIGS. 6-7 and 8, repairs fibrocartilage like menisci with oriented fibers for hoop strength. The hyaline cartilage module, shown in FIGS. 4-5 and 9, replaces damaged cartilage with a low-friction implant, adhering to subchondral bone.

Veterinary and osteochondral repair modules adapt the system for animals and cartilage-bone defects, respectively, using similar hydrogel formulations and delivery methods. The system's biocompatible hydrogel, minimally invasive delivery, and in situ polymerization offer a permanent, high-strength implant surpassing native tissue performance.

### CONCLUSION

The Cartilage Restoration System provides a method, composition, and system for permanently restoring, reinforcing, and replacing fibrocartilaginous and cartilaginous tissues, such as intervertebral discs, knee menisci, labra, and articular cartilage, across human and veterinary orthopedic applications, including spinal fusion. The invention employs an injectable hydrogel that forms a durable, non-degradable implant through in situ polymerization, utilizing a variety of crosslinking methods to ensure rapid and versatile curing. This implant surpasses the mechanical strength of native tissues, offering superior load-bearing performance and seamless chemical and mechanical integration with surrounding tissues to effectively seal defects and support long-term functionality. The system's biocompatibility minimizes immune response risks, and its design supports regulatory compliance for clinical use.

A specialized delivery apparatus enables minimally invasive administration, accommodating diverse anatomical sites and facilitating applications like disc regeneration, meniscal and labral repair, hyaline cartilage replacement, osteochondral repair, and veterinary joint restoration. Modular configurations tailor the system to specific tissue needs, enhancing joint stability and restoring biomechanical function. By providing immediate structural support, enduring durability, and adaptability for both human and animal use, the Cartilage Restoration System reduces reliance on invasive surgical interventions, offering a comprehensive solution for improving orthopedic outcomes across a wide range of clinical scenarios.

1. A method for restoring, reinforcing, replacing, or prophylactically enhancing the biomechanical performance of a load-bearing cartilaginous tissue in a human or veterinary subject, the method comprising:
(a) delivering, by a minimally invasive delivery procedure, into a target load-bearing fibrocartilaginous or cartilaginous tissue of a human or veterinary subject, an injectable biomaterial precursor comprising:
(i) one or more biocompatible hydrogel-forming base polymers;
(ii) one or more natural polymer additives or extracellular matrix-mimicking components;
(iii) one or more crosslinking systems;
(iv) one or more reinforcing agents; and
(v) optionally, one or more adhesive or bioactive components;
(b) polymerizing the injectable hydrogel precursor in situ under physiologic conditions or in response to one or more crosslinking mechanisms selected from thermal, chemical, photochemical, enzymatic, ionic, or combinations thereof, to form a permanent, non-degradable, load-bearing structural matrix within the target tissue; and
(c) chemically and mechanically integrating the permanent, non-degradable structural matrix with native tissue to restore, reinforce, or replace the biomechanical function of the target tissue.
wherein the permanent, non-degradable structural matrix provides immediate and long-term structural support through chemical and mechanical integration with native tissue to permanently restore, reinforce, or replace the biomechanical function of the target tissue while increasing its load-bearing capacity beyond that of the corresponding native tissue.

2. The method of claim 1, wherein the target tissue comprises an intervertebral disc, and the permanent, non-degradable structural matrix is configured to restore a degenerated intervertebral disc or prophylactically reinforcing a non-degenerated intervertebral disc by chemically and mechanically integrating with native disc tissue to provide immediate and long-term structural support.

3. The method of claim 2, wherein the permanent, non-degradable structural matrix is configured to restore or maintain the biomechanical function of the intervertebral disc by reinforcing the nucleus pulposus, annulus fibrosus, or both, while preserving physiologic load transfer through chemical and mechanical integration with the native disc tissue and increasing the load-bearing biomechanical performance of the intervertebral disc.

4. The method of claim 3, wherein the target intervertebral disc comprises an annular tear, and the permanent, non-degradable structural matrix chemically and mechanically integrates with the annulus fibrosus to reinforce the annular tear and provide long-term structural support to the intervertebral disc.

5. The method of claim 1, wherein the target tissue comprises a enhanced load-bearing cartilaginous tissue, and the permanent, non-degradable structural matrix chemically and mechanically integrates with native tissue to restore, reinforce, or replace the biomechanical function of the target tissue while providing long-term structural support.

6. The method of claim 5, wherein the permanent, non-degradable structural matrix forms an articulating surface that chemically and mechanically integrates with the surrounding native cartilaginous tissue to provide durable load-bearing support while increasing the biomechanical load-bearing performance of the articulating surface and facilitating long-term joint function.

7. The method of claim 6, wherein the permanent, non-degradable structural matrix chemically and mechanically integrates with the adjacent cartilaginous tissue and underlying subchondral bone to provide stable fixation and long-term enhanced load-bearing structural support.

8. The method of claim 1, wherein the target tissue comprises a fibrocartilaginous tissue selected from the group consisting of a meniscus, a glenoid labrum, an acetabular labrum, and a triangular fibrocartilage complex, wherein the permanent, non-degradable structural matrix chemically and mechanically integrates with the native fibrocartilaginous tissue to restore, reinforce, or replace its biomechanical function while providing long-term structural support.

9. The method of claim 1, wherein the subject is a veterinary subject, and the target tissue comprises a enhanced load-bearing fibrocartilaginous or cartilaginous tissue, wherein the permanent, non-degradable structural matrix chemically and mechanically integrates with the native tissue to restore, reinforce, or replace its biomechanical function while providing long-term structural support.

10. The method of claim 1, wherein the target tissue comprises an osteochondral defect, and the permanent, non-degradable structural matrix chemically and mechanically integrates with the native cartilaginous tissue and the underlying subchondral bone to restore, reinforce, or replace the biomechanical function of the osteochondral unit while providing long-term structural support.

11. The method of claim 1, wherein the permanent, non-degradable structural matrix remains integrated with the native fibrocartilaginous or cartilaginous tissue under physiologic load to provide sustained biomechanical reinforcement without requiring surgical fixation.

12. The method of claim 1, wherein the permanent, non-degradable structural matrix is configured to remain within the target fibrocartilaginous or cartilaginous tissue as a permanent load-bearing implant that maintains long-term biomechanical function through chemical and mechanical integration with the native tissue while increasing the biomechanical load-bearing performance of the treated tissue beyond that of the corresponding native tissue.

13. A hydrogel composition for permanent tissue restoration, comprising:
(a) a synthetic or natural base polymer at 10-15% w/v, selected from the group consisting of polyethylene glycol diacrylate, poly(vinyl alcohol), methacrylated compounds, or other hydrogel-forming polymers;
(b) a natural polymer additive at 1-2% w/v, selected from the group consisting of hyaluronic acid derivatives, collagen, chitosan, or other extracellular matrix mimics;
(c) a crosslinking agent at 0.1-0.5% w/v, configured for in situ polymerization via a trigger selected from the group consisting of thermal, chemical, photochemical, enzymatic, ionic, or other triggers, or combinations thereof;
(d) reinforcing agents providing a compressive strength greater than 3 MPa, validated by ASTM-compliant testing;
(e) optionally, adhesive components or bioactive molecules to enhance tissue integration; and
(f) biocompatible properties suitable for long-term implantation, with manufacturing processes ensuring sterility and scalability.

14. The hydrogel composition of claim 13, wherein the crosslinking agent includes transglutaminase for enzymatic polymerization without external energy sources.

15. A system for permanent fibrocartilage restoration, comprising:
(a) the hydrogel composition of claim 13; and
(b) a delivery apparatus comprising a dual-chamber syringe or catheter configured for percutaneous injection, mixing hydrogel precursors at ejection, and optionally including a light source for photochemical polymerization, a heating element for thermal polymerization, or an electrode for initiating polymerization.

16. The system of claim 15, wherein the implant reinforces existing tissue without altering its shape, forming a protective layer with a load-bearing capacity at least three times that of native tissue, validated by finite element analysis.
